(19) United States
(12) Patent Application Publication
(10) Pub. No.: US 2016/0376563 A1
(43) Pub. Date: Dec. 29, 2016

(54) Methods and Compositions for Treating Obesity

(71) Applicants: Kim D. JANDA, La Jolla, CA (US); THE SCRIPPS RESEARCH INSTITUTE, La Jolla, CA (US)

(72) Inventor: Kim D. Janda, La Jolla, CA (US)

(21) Appl. No.: 14/901,794

(22) PCT Filed: Jul. 1, 2014
(86) PCT No.: PCT/US14/45019

§ 371 Date: Dec. 29, 2015

Related U.S. Application Data
(60) Provisional application No. 61/842,091, filed on Jul. 2, 2013.

(51) Int. Cl. C12N 9/00

(52) U.S. Cl. CPC C12N 9/0002

(57) ABSTRACT

The present invention provides anti-ghrelin antibodies or antigen-binding molecules that are capable of degrading ghrelin and inhibiting ghrelin-mediated cellular activities. Also provided in the invention are therapeutic applications of combinations of these antibodies, e.g., to treat or prevent obesity.

### BACKGROUND OF THE INVENTION

Obesity is a chronic, costly, and globally prevalent condition, with excess caloric intake a suspected etiologic factor. Approximately I billion people worldwide are overweight or obese (body mass index=25-29.9 or >30 kg/m², respectively). These conditions are associated with significant morbidity and mortality and for which new treatments are needed. Nonsurgical treatments of obesity are modestly efficacious, and weight loss maintenance is hampered by anti-famine homeostatic mechanisms.

Human ghrelin is a 28-amino acid acylated peptide (Kojima et al., Nature 402:656-60, 1999). It is released mainly from endocrine cells of the stomach and upper gastrointestinal tract but also expressed in testes, kidney, pituitary, pancreas, lymphocytes, and brain. Gastric ghrelin has been identified as an indicator of energy insufficiency and anabolic modulator of energy homeostasis. Human studies have found a preprandial rise and postprandial decline in plasma ghrelin levels, consistent with a role for ghrelin in hunger and meal initiation. Indeed, circulating ghrelin levels are increased by food deprivation and decreased by meals, glucose load, insulin, and somatostatin. Pharmacological increases in ghrelin trigger food intake in rats or humans and decrease energy expenditure and the relative utilization of fat as an energy substrate, leading to weight gain and adiposity with chronic central administration.

### SUMMARY OF THE INVENTION

In one aspect, the present invention provides monoclonal antibodies or antigen-binding molecules which can bind to ghrelin with the same binding specificity as the antibody produced by the hybridoma cell line with ATCC™ deposit number PTA-120177. In various embodiments, the antibody is a catalytic antibody capable of degrading ghrelin. In some preferred embodiments, the antibody is capable of binding to human ghrelin.

Some antibodies or antigen-binding molecules of the invention contain grafted complementarity determining regions (CDRs) of the heavy chain and/or CDRs of the light chain of the antibody produced by the hybridoma cell line with ATCC™ deposit number PTA-120177. Some other antibodies contain the same variable regions as that of the antibody produced by the hybridoma cell line with ATCC™ deposit number PTA-120177. Also encompassed by the invention is the antibody produced by hybridoma cell line with ATCC™ deposit number PTA-120177.

In some embodiments, the antibody is a mouse antibody. Some other embodiments of the invention are directed to chimeric antibodies. For example, such antibodies can contain mouse variable region sequences and human constant region sequences. In some other embodiments, the anti-ghrelin antibodies of the invention are humanized or entirely human. Some antibodies of the invention are full length. Some other anti-ghrelin antibodies of the invention are antigen-binding fragments derived from a full length antibody, e.g., scFv fragments, Fv fragments, Fd fragments, Fab fragments or F(ab′)₂ fragments.

In a related aspect, the invention provides isolated or recombinant polynucleotides which encode a polypeptide that contain the variable region of the heavy chain or the variable region of the light chain of the anti-ghrelin antibody or antigen-binding molecule of the invention (e.g., antibody produced by the hybridoma cell line with ATCC™ deposit number PTA-120177). Also provided in the invention are hybrid cell lines which produce a monoclonal antibody which is specifically reactive with ghrelin and which has the binding specificity of the antibody produced by hybridoma cell line with ATCC™ deposit number PTA-120177. The invention additionally provides pharmaceutical compositions that contain a therapeutically effective amount of an anti-ghrelin antibody or antigen-binding fragment of the invention.

In another aspect, the invention provides methods of inhibiting or slowing weight gain in a subject. The methods entail administering to the subject a pharmaceutical composition comprising an antibody or antigen-binding fragment that binds to ghrelin with the same binding specificity as that of the antibody produced by the hybridoma cell line with ATCC™ deposit number PTA-120177. In some preferred embodiments, the administered antibody is a catalytic antibody capable of degrading ghrelin. In some embodiments, the subject to be treated is a human. In various embodiments, the administered antibody can be a murine antibody, a chimeric antibody, a humanized antibody, or a human antibody.

In another aspect, the invention provides methods for treating obesity in a subject. The methods involve administering to the subject a pharmaceutical composition containing an antibody or antigen-binding fragment that binds to ghrelin with the same binding specificity as that of the antibody produced by the hybridoma cell line with ATCC™ deposit number PTA-120177. In some embodiments, the administered antibody is a catalytic antibody capable of degrading ghrelin, e.g., human ghrelin. Some preferred embodiments of the invention are directed to treating obesity in human subjects. In various embodiments, the antibody to be administered to the subject can be a murine antibody, a chimeric antibody, a humanized antibody, or a human antibody.

### DESCRIPTION OF THE DRAWINGS

FIG. 1 shows structures of ghrelin-derived peptides 1-10 synthesized for the generation, specificity and kinetic characterization of catalytic mAbs. Note that rat and human ghrelins are both 28 amino acid residues in length and differ only at positions 11 and 12. Thus, the first 5 amino acid residues of rat and human ghrelins, ghrelin—(1-5), are identical.

FIG. 2 shows results of kinetic evaluation of catalyzed ghrelin hydrolysis by antibody GHR-11E11. (Left) Catalysis of ghrelin hydrolysis by addition of 1 μM GHR-11E11 to varying concentrations of substrate 3 (0.4-50 μM) at 37° C. (Right) Dose-response plot of inhibition of ghrelin hydrolysis by 1 μM GHR-11E11 with varying inhibitor 1 concentrations; IC50: 21.3 μM.

FIG. 3 shows the rate of energy expenditure (heat, Top Left), respiratory exchange ratio (RER) (Top Right), the rates of oxygen consumption (VO2) (Middle Left) and carbon dioxide production (VCO2) (Middle Right), and rates of horizontal and vertical motor activity (Bottom) in food-deprived, antibody-treated, adult male C57BL/6J mice tested in open-circuit indirect calorimetry chambers. Data are expressed in 2 h bins as M±SEM across the 12-h light cycle. Mice received i.v. administration (i.v. 50 mg/kg) of a catalytic antibody against ghrelin (n=8, GHR-11E11) or of an isotype-matched nicotine control Ab (n=9, NIC-1 9D9) before data collection; *, P<0.05 vs. control Ab-treated mice.

FIG. 4 shows food intake in 24-h food-deprived adult male C57BL/6J mice that had received i.v. administration (i.v. 50 mg/kg) of a catalytic antibody against ghrelin (n=8, GHR-11E11) or of an isotype-matched nicotine control Ab (n=9, NIC-1 9D9) 24 h earlier. Data express M±SEM cumulative food intake across 6 h of refeeding beginning from the light cycle onset. *, P<0.05 vs. control Ab-treated mice.

FIG. 5 shows the rate of energy expenditure (heat) (Top Left), respiratory exchange ratio (RER) (Top Right), rates of oxygen consumption (VO2) (Bottom Left), and carbon dioxide production (VCO2) (Bottom Right) in adult male C57BL/6J mice residing in open-circuit indirect calorimetry chambers. Data were collected in a 24-h food-deprived state (“Unfed”) and during 6 h of refeeding on chow. Data are expressed in 1 h bins as M±SEM. Mice had received i.v. administration (i.v. 50 mg/kg) of a catalytic antibody against ghrelin (n=8, GHR-11E11) or of an isotype-matched nicotine control Ab (n=9, NIC-1 9D9) 24 h before data collection; *, P<0.05 vs. control Ab-treated mice.

### DETAILED DESCRIPTION OF THE INVENTION

### I. Overview

The present invention is predicated in part on the development by the present inventor of novel catalytic antibodies which enable pharmacological degradation of ghrelin and reduction of ghrelin's biological effects. The antibodies are capable of both sequestering and degrading the ester moiety of ghrelin, and thus essentially deactivating ghrelin. Specifically, the anti-ghrelin immunopharmacotherapy is based on catalytic antibodies that hydrolyze the Ser-3 octanoate ester moiety of ghrelin. As detailed herein, the inventor generated antibodies that hydrolyze the octanoyl moiety of ghrelin to form des-acyl ghrelin. One of the identified antibody catalysts, antibody GHR-11E11 (ATCC Deposit Designation PTA-120177), was found to display a second-order rate constant of 18 M⁻1·s⁻¹ for the hydrolysis of ghrelin to des-acyl ghrelin. I.v. administration of GHR-11E11 (50 mg/kg) maintained a greater metabolic rate in fasting C57BL/6J mice as compared with mice receiving a control antibody and suppressed 6-h refeeding after 24 h of food deprivation. Indirect respiratory measures of metabolism after refeeding and relative fuel substrate utilization were unaffected. The results support the hypothesis that acylated ghrelin stimulates appetite and curbs energy expenditure during deficient energy intake, whereas des-acyl ghrelin does not potently share these functions. Catalytic anti-ghrelin antibodies might thereby adjunctively aid consolidation of caloric restriction-induced weight loss and might also be therapeutically relevant to Prader-Willi syndrome, characterized after infancy by hyperghrelinemia, hyperphagia, and obesity.

The inventor's studies demonstrated that passive immunopharmacotherapy with a catalytic anti-ghrelin antibody such as GHR-11E11 can both decrease the serum ghrelin/des-acyl ghrelin ratio and modulate energy homeostasis. Ghrelin, an endogenous peptide ligand for the GHSRIa receptor released into circulation from the stomach, is posttranslationally acylated by the addition of octanoic acid to the Ser-3 residue. This modification is critical for ghrelin's active transport across the blood-brain barrier and potent GHSR1a activity, Whereas infusion of acylated ghrelin acutely stimulates food intake and subjective hunger and chronic administration causes weight gain, administration of the hydrolytic degradation product des-acyl ghrelin either does not stimulate appetite or does so less effectively than acylated ghrelin. Administration of an antibody of the present invention can both bind and degrade ghrelin to its des-acyl form maintained a relatively increased metabolic rate in fasting mice suppressed refeeding after food deprivation.

In accordance with results obtained from these studies, the invention provides catalytic anti-ghrelin antibodies and related pharmaceutical compositions. The antibodies and pharmaceutical compositions containing the antibodies are useful as therapeutic or prophylactic agents in treating obesity and preventing undesired weight gain. The following sections provide guidance for making and using the compositions of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3PrdP ed., 2002); and A Dictionary of Biology (Oxford Paperback Reference), Martin and Hine (Eds.), Oxford University Press (4PthP ed., 2000). In addition, the following definitions are provided to assist the reader in the practice of the invention.

The term “antibody” and “antigen-binding molecule” is used to denote polypeptide chain(s) which exhibit a strong monovalent, bivalent or polyvalent binding to a given epitope or epitopes. Unless otherwise noted, antibodies or antigen-binding molecules of the invention can have sequences derived from any vertebrate, camelid, avian or pisces species. They can be generated using any suitable technology, e.g., hybridoma technology, ribosome display, phage display, gene shuffling libraries, semi-synthetic or fully synthetic libraries or combinations thereof. As detailed herein, antibodies or antigen-binding molecules of the invention include intact antibodies, antigen-binding polypeptide chains and other designer antibodies (see, e.g., Serafini, J. Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The VH and VL regions of an antibody can be further subdivided into regions of hypervariability, also termed complementarity determining regions (CDRs), which are interspersed with the more conserved framework regions (FRs). Each VH and VL is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

Amino acids from the variable regions of the mature heavy and light chains of immunoglobulins are designated Hx and Lx respectively, where x is a number designating the position of an amino acid according to the scheme of Kabat et al, supra. Kabat et al. list many amino acid sequences for antibodies for each subgroup, and lists the most commonly occurring amino acid for each residue position in that subgroup to generate a consensus sequence. Kabat et al. use a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat et al. by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention.

Antibody or antigen-binding molecule also includes antibody fragments which contain the antigen-binding portions of an intact antibody that retain capacity to bind the cognate antigen. Examples of such antibody fragments include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CHI domains; (iv) an Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242:423-426, 1988; and Huston et al., Proc. Natl. Acad. Sci. USA 85:5879-5883, 1988.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

The various antibodies or antigen-binding fragments described herein can be produced by enzymatic or chemical modification of the intact antibodies, or synthesized de novo using recombinant DNA methodologies (e.g., single chain Fv), or identified using phage display libraries (see, e.g., McCafferty et al., Nature 348:552-554, 1990). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries or ribosome display libraries, gene shuffled libraries. Such libraries can be constructed from synthetic, semi-synthetic or nave and immunocompetent sources.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, as shown in the Examples below, a mouse anti-ghrelin antibody can be modified by replacing its constant region with the constant region from a human immunoglobulin. Due to the replacement with a human constant region, the chimeric antibody can retain its specificity in recognizing human ghrelin while having reduced antigenicity in human as compared to the original mouse antibody.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlanl, Molec. Immun., 28:489-498, 1991; and Padlan2, Molec. Immun., 31:169-217, 1994.

The term “catalytic antibody” or “catalytic antibody” refers to an antibody capable of inhibiting or suppressing activation or biological activities of the cognate antigen (e.g., a receptor) to which the antibody specifically binds. For example, an anti-ghrelin catalytic antibody binds to and degrades ghrelin. As a result, cellular or signaling activities mediated by ghrelin are inhibited or suppressed.

Binding specificity of an antibody or antigen-binding molecule refers to the ability of the combining site of an individual antibody or antigen-binding molecule to react with only one antigenic determinant. The combining site of a typical antibody is located in the Fab portion of the molecule and is constructed from the hypervariable regions of the heavy and light chains. Binding affinity is the strength of the reaction between a single antigenic determinant and a single combining site on the antibody or antigen-binding molecule. It is the sum of the attractive and repulsive forces operating between the antigenic determinant and the combining site. Affinity is the equilibrium constant that describes the antigen-antibody reaction.

The phrase “specifically (or selectively) bind to” refers to a binding reaction between an antibody or antigen-binding molecule (e.g., an anti-ghrelin antibody) and a cognate antigen (e.g., a human ghrelin polypeptide) in a heterogeneous population of proteins and other biologics. The phrases “an antibody recognizing an antigen” and “an antibody specific for an antigen” are used interchangeably herein with the term “an antibody which binds specifically to an antigen”.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acids Res. 19:5081, 1991; Ohtsuka et al., J Biol. Chem. 260:2605-2608, 1985; and Rossolini et al., Mol. Cell. Probes 8:91-98, 1994).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “treating” includes the administration of compounds or agents to prevent or delay the onset of the symptoms, complications, or biochemical indicia of a disease (e.g., a tumor), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

III. Catalytic Anti-Ghrelin Antibodies Derived from ATCC™ Deposit Number PTA-120177

The invention provides monoclonal antibodies or antigen-binding molecules that specifically bind to and catalyze hydrolysis of ghrelin protein or peptide thereof. These anti-ghrelin agents are capable of suppressing ghrelin mediated signaling or cellular activities, e.g., ghrelin induced food intake as described in the Examples below. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, N.Y., 1998; Kohler & Milstein, Nature 256:495-497, 1975; Kozbor et al., Immunology Today 4:72, 1983; and Cole et al., pp. 77-96 in Monoclonal Antibodies and Cancer Therapy, 1985.

Some of the anti-ghrelin antibodies of the invention are catalytic monoclonal antibodies that are directly derived from the anti-ghrelin antibody clone GHR-11E11 which is produced by the hybridoma cell line of ATCCTM deposit number PTA-120177. Activities of antibody GHR-11E11 is described in the Examples below. Various monoclonal antibodies or antigen-binding fragments with similar binding and catalytic activities can be derived from the exemplified antibody. These antibodies can be generated by any technique for producing monoclonal antibody well known in the art, e.g., viral or oncogenic transformation of B lymphocytes. One animal system for preparing hybridomas is the murine system. Hybridoma production in the mouse is a very well-established procedure. As illustrated in the Examples below, catalytic monoclonal anti-ghrelin antibodies can be generated by reactive immunization of a non-human animal (e.g., mouse) with a carrier protein conjugated transition state analog of ghrelin. B cells isolated from the animal are then fused to myeloma cells to generate antibody-producing hybridomas. Monoclonal mouse anti-ghrelin antibodies can be obtained by screening the hybridomas in an ELISA assay using a ghrelin polypeptide or fusion protein. Immunization protocols and techniques for isolation of immunized splenocytes for fusion are known in the art. Fusion partners (e.g., murine myeloma cells) and fusion procedures are also well known in the art, e.g., Harlow & Lane, supra. Other than binding activities, the anti-ghrelin antibodies derived from antibody GHR-11E11 should also have similar or enhanced catalytic activities against ghrelin. Catalytic activities of the anti-ghrelin antibodies of the invention can be assessed by standard biochemistry assays. For example, catalytic activities of the antibodies can be examined via analytic HPLC methods using a number of ghrelin derived peptide substrates exemplified herein.

A typical intact antibody interacts with target antigen predominantly through amino acid residues that are located in the six heavy and light chain complimentarity determining regions (CDR's). Typically, the anti-ghrelin antibodies of the invention have at least one of their heavy chain CDR sequences or light chain CDR sequences identical to the corresponding CDR sequences of anti-ghrelin antibody clone GHR-11E11. Some of these anti-ghrelin antibodies of the invention have the same binding specificity as that of the exemplified mouse anti-ghrelin antibody (clone GHR-11E11) disclosed in the Examples below. These antibodies can compete with the mouse anti-ghrelin antibody (clone GHR-11E11) for binding to ghrelin. Some anti-ghrelin antibodies of the invention have all CDR sequences in their variable regions of the heavy chain and light chain respectively identical to the corresponding CDR sequences anti-ghrelin antibody clone GHR-11E11.

In addition to having CDR sequences respectively identical to the corresponding CDR sequences of the mouse anti-ghrelin antibody (clone GHR-11E11), some of the anti-ghrelin antibodies of the invention have their entire heavy chain and light chain variable region sequences respectively identical to the corresponding variable region sequences of the mouse antibody clone GHR-11E11. In some other embodiments, other than the identical CDR sequences, the antibodies contain amino acid residues in the framework portions of the variable regions that are different from the corresponding amino acid residues of mouse anti-ghrelin antibody clone GHR-11E11 (e.g., some of the humanized anti-ghrelin antibodies described below). Nevertheless, these antibodies typically have their entire variable region sequences that are substantial identical (e.g., 75%, 85%, 90%, 95%, or 99%) to the corresponding variable region sequences of mouse anti-ghrelin antibody clone GHR-11E11.

The anti-ghrelin antibodies of the invention can be an intact antibody which contains two heavy chains and two light chains. They can also be antigen-binding molecules of an intact antibody or single chain antibodies. The anti-ghrelin antibodies of the invention include antibodies produced in a non-human animal (e.g., the mouse anti-ghrelin antibody clone GHR-11E11). They also include modified antibodies which are modified forms of the mouse anti-ghrelin antibody clone GHR-11E11. Often, the modified antibodies are recombinant antibodies which have similar or improved properties relative to that of the exemplified mouse antibody. For example, the mouse anti-ghrelin antibody exemplified in the Examples below can be modified by deleting the constant region and replacing it with a different constant region that can lead to increased half-life, e.g., serum half-life, stability or affinity of the antibody. The modified antibodies can be created, e.g., by constructing expression vectors that include the CDR sequences from the mouse antibody grafted onto framework sequences from a different antibody with different properties (Jones et al. 1986, Nature 321, 522-525). Such framework sequences can be obtained from public DNA databases (e.g., from www.kabatdatabase.com).

### IV. Modified Anti-Ghrelin Catalytic Antibodies

Some embodiments of the invention are directed to modified antibodies that are based on or derived from mouse anti-ghrelin antibody GHR-11E11 which is produced by hybridoma cell line of ATCC™ deposit number PTA-120177. These include, e.g., chimeric, humanized and human anti-ghrelin catalytic antibodies. Relative to antibody GHR-11E11, these modified antibodies have similar or improved binding specificity and/or catalytic activities. They also have substantially reduced antigenicity when used in vivo in a non-mouse subject, e.g., a human subject. Some of the modified antibodies are chimeric antibodies which contain partial human immunoglobulin sequences (e.g., constant regions) and partial non-human immunoglobulin sequences (e.g., the mouse anti-ghrelin antibody variable region sequences of mouse anti-ghrelin antibody clone GHR-11E11). Some other modified antibodies are humanized antibodies. Generally, a humanized antibody has one or more amino acid residues introduced into it from a source that is non-human. Methods for humanizing non-human antibodies are well known in the art, e.g., U.S. Pat. Nos. 5,585,089 and 5,693,762; Jones et al., Nature 321: 522-25, 1986; Riechmann et al., Nature 332: 323-27, 1988; and Verhoeyen et al., Science 239: 1534-36, 1988. These methods can be readily employed to generate humanized anti-ghrelin antibodies of the invention by substituting at least a portion of a CDR from a non-human anti-ghrelin antibody for the corresponding regions of a human antibody. In some embodiments, the humanized anti-ghrelin antibodies of the invention have all three CDRs in each immunoglobulin chain from the mouse anti-ghrelin antibody clone GHR-11E11 grafted into corresponding human framework regions.

The anti-ghrelin antibodies described above can undergo non-critical amino-acid substitutions, additions or deletions in both the variable and constant regions without loss of binding specificity or effector functions, or intolerable reduction of binding affinity. Usually, antibodies incorporating such alterations exhibit substantial sequence identity to a reference antibody (e.g., the mouse anti-ghrelin antibody clone GHR-11E11) from which they were derived. For example, the mature light chain variable regions of some of the anti-ghrelin antibodies of the invention have at least 75% or at least 85% sequence identity to the sequence of the mature light chain variable region of the exemplified mouse anti-ghrelin antibodies. Similarly, the mature heavy chain variable regions of the antibodies typically show at least 75% or at least 85% sequence identity to the sequence of the mature heavy chain variable region of the exemplified anti-ghrelin antibodies. Some of the modified anti-ghrelin antibodies have the same specificity and increased affinity compared with the exemplified mouse anti-ghrelin antibodies. Usually, the affinity of the modified anti-ghrelin antibodies is within a factor of 2, 5, 10 or 50 of the reference mouse anti-ghrelin antibody.

Some of the anti-ghrelin antibodies of the invention are chimeric (e.g., mouse/human) antibodies which are made up of regions from a non-human anti-ghrelin catalytic antibody together with regions of human antibodies. For example, a chimeric H chain can comprise the antigen binding region of the heavy chain variable region of the mouse anti-ghrelin antibody exemplified herein (e.g., GHR-11E11) linked to at least a portion of a human heavy chain constant region. This chimeric heavy chain may be combined with a chimeric L chain that comprises the antigen binding region of the light chain variable region of the exemplified mouse anti-ghrelin antibody (e.g., GHR-11E11) linked to at least a portion of the human light chain constant region.

Chimeric anti-ghrelin antibodies of the invention can be produced in accordance with methods known in the art. For example, a gene encoding the heavy chain or light chain of a murine anti-ghrelin antibody or antigen-binding molecule can be digested with restriction enzymes to remove the murine Fc region, and substituted with the equivalent portion of a gene encoding a human Fc constant region. Expression vectors and host cells suitable for expression of recombinant antibodies and humanized antibodies in particular, are well known in the art. Vectors expressing chimeric genes encoding anti-ghrelin immunoglobulin chains can be constructed using standard recombinant techniques, e.g., Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (Ringbou, ed., 2003). Human constant region sequences can be selected from various reference sources, including but not limited to those listed in Kabat et al., Sequences of Proteins of Immunological Interest, Fifth Edition, U.S. Department of Health and Human Services, U.S. Government Printing Office, 1991. More specific teachings of producing chimeric antibodies by DNA recombination have also been taught in the art, e.g., Robinson et al., International Patent Application PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science 240:1041-1043, 1988; Liu et al., PNAS 84:3439-3443, 1987; Liu et al., J. Immunol. 139:3521-3526, 1987; Sun et al., PNAS 84:214-218, 1987; Nishimura et al., Canc. Res. 47:999-1005, 1987; Wood et al., Nature 314:446-449, 1985; Shaw et al., J. Natl. Cancer Inst. 80:1553-1559, 1988.

Chimeric antibodies which have the entire variable regions from a non-human antibody can be further humanized to reduce antigenicity of the antibody in human. This is typically accomplished by replacing certain sequences or amino acid residues in the Fv variable regions (framework regions or non-CDR regions) with equivalent sequences or amino acid residues from human Fv variable regions. These additionally substituted sequences or amino acid residues are usually not directly involved in antigen binding. More often, humanization of a non-human antibody proceeds by substituting only the CDRs of a non-human antibody (e.g., the mouse anti-ghrelin antibodies exemplified herein) for the CDRs in a human antibody. In some cases, this is followed by replacing some additional residues in the human framework regions with the corresponding residues from the non-human donor antibody. Such additional grafting is often needed to improve binding to the antigen. This is because humanized antibodies which only have CDRs grafted from a non-human antibody can have less than perfect binding activities as compared to that of the non-human donor antibody. Thus, in addition to the CDRs, humanized anti-ghrelin antibodies of the invention can often have some amino acids residues in the human framework region replaced with corresponding residues from the non-human donor antibody (e.g., the mouse antibody exemplified herein). Methods for generating humanized antibodies by CDR substitution, including criteria for selecting framework residues for replacement, are well known in the art. For example, in addition to the above noted art relating to producing chimeric antibodies, additional teachings on making humanized antibodies are provided in, e.g., Winter et al., UK Patent Application GB 2188638A (1987), U.S. Pat. No. 5,225,539; Jones et al., Nature 321:552-525, 1986; Verhoeyan et al., Science 239:1534, 1988; and Beidler et al., J. Immunol. 141:4053-4060, 1988. CDR substitution can also be carried out using oligonucleotide site-directed mutagenesis as described in, e.g., WO 94/10332 entitled “Humanized Antibodies to Fc Receptors for Immunoglobulin G on Human Mononuclear Phagocytes.”

The chimeric or humanized anti-ghrelin antibodies of the invention may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

In addition to chimeric or humanized anti-ghrelin antibodies, also included in the invention are fully human antibodies that exhibit the same binding specificity and comparable or better binding affinity. For example, the human anti-ghrelin antibodies can have the same or better binding characteristics (e.g., binding specificity and/or binding affinity) relative to that of a reference nonhuman anti-ghrelin antibody, e.g., mouse anti-ghrelin antibody clone GHR-11E11. The reference nonhuman antibody can be the mouse anti-ghrelin antibody produced by the hybridoma cell line with ATCC™ deposit number PTA-120177. Compared to the chimeric or humanized antibodies, the human anti-ghrelin antibodies of the invention have further reduced antigenicity when administered to human subjects.

The human anti-ghrelin antibodies can be generated using methods that are known in the art. For example, an in vivo method for replacing a nonhuman antibody variable region with a human variable region in an antibody while maintaining the same or providing better binding characteristics relative to that of the nonhuman antibody has been disclosed in U.S. patent application Ser. No. 10/778,726 (Publication No. 20050008625). The method replies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

The anti-ghrelin antibodies or antigen-binding molecules of the invention also include single chain antibodies, bispecific antibodies and multi-specific antibodies. In some embodiments, the antibodies of the invention are single chain antibodies. Single chain antibodies contain in a single stably-folded polypeptide chain the antigen-binding regions from both the heavy chain and the light chain. As such, single chain antibodies typically retain the binding specificity and affinity of monoclonal antibodies but are of considerably small size than classical immunoglobulins. For certain applications, the anti-ghrelin single chain antibodies of the invention may provide many advantageous properties as compared to an intact anti-ghrelin antibody. These include, e.g., faster clearance from the body, greater tissue penetration for both diagnostic imaging and therapy, and a significant decrease in immunogenicity when compared with mouse-based antibodies. Other potential benefits of using single chain antibodies include enhanced screening capabilities in high throughput screening methods and the potential for non-parenteral application. Single chain anti-ghrelin antibodies of the invention can be prepared using methods that have been described in the art. Examples of such techniques include those described in U.S. Pat. Nos. 4,946,778 and 5,258,498; Huston et al., Methods in Enzymology 203:46-88, 1991; Shu et al., Proc. Natl. Acad. Sci. USA 90:7995-7999, 1993; and Skerra et al., Science 240:1038-1040, 1988.

### V. Polynucleotides, Vectors and Host Cells for Producing Anti-Ghrelin Antibodies

The invention provides substantially purified polynucleotides (DNA or RNA) which encode polypeptides comprising segments or domains of the anti-ghrelin antibody chains or antigen-binding molecules described above. Some of the polynucleotides of the invention comprise the nucleotide sequence encoding the heavy chain variable region of a described anti-ghrelin antibody, e.g., mouse anti-ghrelin antibody GHR-11E11 which is produced by hybridoma cell line of ATCC™ deposit number PTA-120177. They can alternatively or additionally comprise the nucleotide sequence encoding the light chain variable region of the described anti-ghrelin antibody. Some other polynucleotides of the invention comprise nucleotide sequences that are substantially identical (e.g., at least 65, 80%, 95%, or 99%) to the nucleotide sequence encoding the heavy chain variable region or light chain variable region of an described anti-ghrelin antibody (e.g., antibody GHR-11E11). Also provided in the invention are polynucleotides which encode at least one CDR region and usually all three CDR regions from the heavy or light chain of a described anti-ghrelin antibody, e.g., mouse anti-ghrelin antibody GHR-11E11. Because of the degeneracy of the code, a variety of nucleic acid sequences will encode each of the immunoglobulin amino acid sequences. When expressed from appropriate expression vectors, polypeptides encoded by these polynucleotides are capable of exhibiting antigen binding capacity.

The polynucleotides of the invention can encode only the variable region sequence of an anti-ghrelin antibody. They can also encode both a variable region and a constant region of the antibody. Some of polynucleotide sequences of the invention encode a mature heavy chain variable region sequence that is substantially identical (e.g., at least 80%, 90%, or 99%) to the mature heavy chain variable region sequence of an anti-ghrelin antibody described herein (e.g., mouse antibody GHR-11E11). Some other polynucleotide sequences encode a mature light chain variable region sequence that is substantially identical to the mature light chain variable region sequence of the described anti-ghrelin antibody. Some of the polynucleotide sequences encode a polypeptide that comprises variable regions of both the heavy chain and the light chain of a disclosed anti-ghrelin antibody, e.g., mouse anti-ghrelin antibody GHR-11E11. Some other polynucleotides encode two polypeptide segments that respectively are substantially identical to the variable regions of the heavy chain and the light chain of one of the disclosed anti-ghrelin antibodies.

The polynucleotide sequences can be produced by de novo solid-phase DNA synthesis or by PCR mutagenesis of an existing sequence (e.g., sequences as described in the Examples below) encoding an anti-ghrelin antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Manila et al., Nucleic Acids Res. 19:967, 1991; and Eckert et al., PCR Methods and Applications 1:17, 1991.

Also provided in the invention are expression vectors and host cells for producing the anti-ghrelin antibodies described above. Various expression vectors can be employed to express the polynucleotides encoding the anti-ghrelin antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-ghrelin polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-ghrelin antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an anti-ghrelin antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-ghrelin antibody sequences. More often, the inserted anti-ghrelin antibody sequences are linked to a signal sequences before inclusion in the vector. Vectors to be used to receive sequences encoding anti-ghrelin antibody light and heavy chain variable domains sometimes also encode constant regions or parts thereof Such vectors allow expression of the variable regions as fusion proteins with the constant regions thereby leading to production of intact antibodies or fragments thereof. Typically, such constant regions are human.

The host cells for harboring and expressing the anti-ghrelin antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-ghrelin polypeptides of the invention. Insect cells in combination with baculovirus vectors can also be used.

In some preferred embodiments, mammalian host cells are used to express and produce the anti-ghrelin polypeptides of the present invention. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-ghrelin antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### VI. Therapeutic Applications and Pharmaceutical Compositions

The anti-ghrelin catalytic antibodies described herein can be employed in many therapeutic or prophylactic applications by degrading the ghrelin protein in subjects suffering from or at the risk of developing a condition or disorder mediated by or associated with ghrelin (e.g., obesity). These include, but are not limited to, decreasing adiposity and treating obesity, preventing the development of obesity, reversing or slowing of weight gain, decreasing feed efficiency, inhibiting restriction induced feeding during low-calorie diet-induced weight loss, and sparing lean body mass in a subject during low-calorie diet-induced weight loss. In therapeutic applications, a composition comprising an anti-ghrelin catalytic antibody or antigen-binding molecule (e.g., a humanized anti-ghrelin antibody) is administered to a subject already affected by a disease or condition caused by or associated with ghrelin (e.g., obesity). The composition contains the antibody or antigen-binding molecule in an amount sufficient to cure, partially arrest, or detectably slow the progression of the condition, and its complications. In prophylactic applications, compositions containing the anti-ghrelin antibodies or antigen-binding molecules are administered to a subject not already suffering from a ghrelin-related disorder. Rather, they are directed to a subject who is at the risk of, or has a predisposition, to developing such a disorder. Such applications allow the subject to enhance the subject's resistance or to retard the progression of a disorder mediated by ghrelin.

The invention provides pharmaceutical compositions comprising the anti-ghrelin antibodies or antigen-binding molecules formulated together with a pharmaceutically acceptable carrier. The compositions can additionally contain other therapeutic agents that are suitable for treating or preventing a given disorder. Pharmaceutically carriers enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the anti-ghrelin antibody is employed in the pharmaceutical compositions of the invention. The anti-ghrelin antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies of the invention employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg.

In various embodiments, a pharmaceutical composition of the invention is usually administered on multiple occasions. Intervals between single dosages can be weekly, monthly or yearly. An exemplary treatment regime entails administration once per every two weeks or once a month or once every 3 to 6 months. Intervals can also be irregular as indicated by measuring blood levels of anti-ghrelin antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### VII. Deposit of Materials

Murine hybridoma GHR 11E11 was deposited with and tested by the American Type Culture Collection, Manassas, Va., USA (ATCC®) on May 6, 2013, and has been assigned the ATCC® Patent Deposit Designation PTA-120177. The deposit provides a cell line that expresses mouse anti-ghrelin antibody GHR 11E11. The deposit was made under the provisions of the Budapest Treaty on the International Recognition of the Deposit of Microorganisms for the Purpose of Patent Procedure and the Regulations thereunder (Budapest Treaty). This assures maintenance of a viable cell culture for 30 years from the date of the deposit. The cell line will be made available by ATCC under the terms of the Budapest Treaty which assures permanent and unrestricted availability of the progeny of the culture to the public upon issuance of the pertinent U.S. patent, and assures availability of the progeny to one determined by the U.S. Commissioner of Patents and Trademarks to be entitled thereto according to 35 U.S.C. §122 and the Commissioner's rules pursuant thereto (including 37 CFR §1.14 with particular reference to 886 OG 638). The assignee of the present application agreed that, subject to 37 CFR 1.808(b), all restrictions imposed by the depositor on the availability to the public of the deposited biological material be irrevocably removed upon the granting of the patent. The assignee of the present application has also agreed that if the cell culture deposits should die or be lost or destroyed when cultivated under suitable conditions, it will be promptly replaced on notification with a viable specimen of the same culture. Availability of the deposit is not to be construed as a license to practice the invention in contravention of the rights granted under the authority of any government in accordance with its patent laws.

### EXAMPLES

### Example 1

### Generation and Screening of Catalytic Anti-Ghrelin Antibody

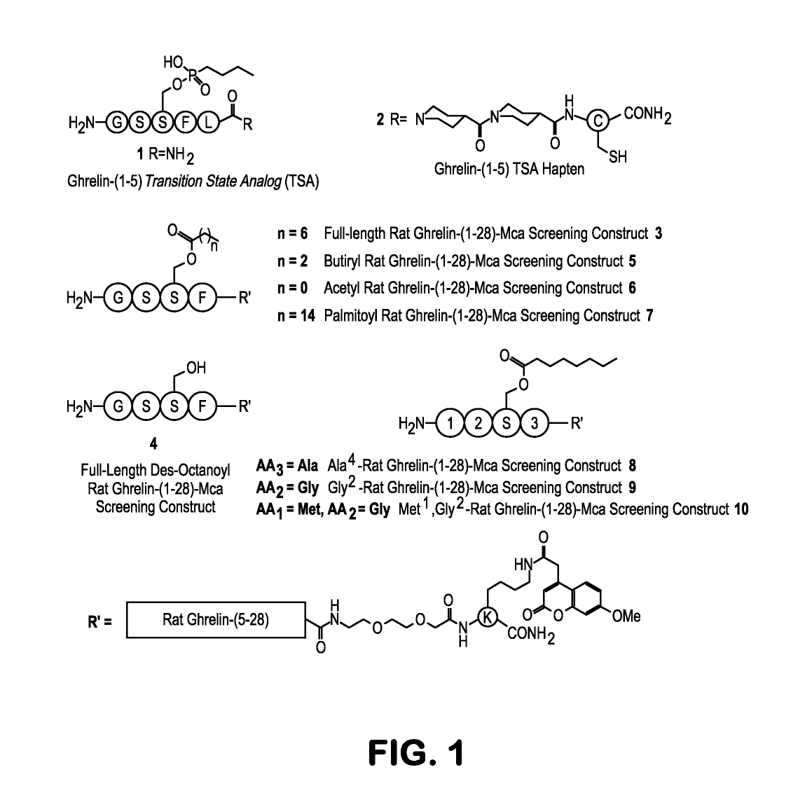

Monoclonal anti-ghrelin antibodies were obtained through immunization of mice with ghrelin phosphonate transition state analog 1, conjugated to the immunogenic carrier protein keyhole limpet hemocyanin (KLH), through a covalent link between the thiol moiety of 1 and an N-maleimidomethyl cyclohexane-1-carboxylate cross-linker, resulting in hapten 2 (FIG. 1). In addition, we extended the hapten with 2 isonipecotic acid (Isn) moieties as a rigid linker to generate a more focused immune response, and a cysteine residue was included to enable a high-yield conjugation to KLH (see above). Hapten 2 was synthesized on solid phase and was coupled to KLH through thioether conjugation chemistry. Immunization of BALB/c mice with the immunoconjugate resulted in a panel of 19 monoclonal catalytic antibodies (mAbs) for analysis. All mAbs were purified from ascites, using ion-exchange and protein G affinity chromatography.

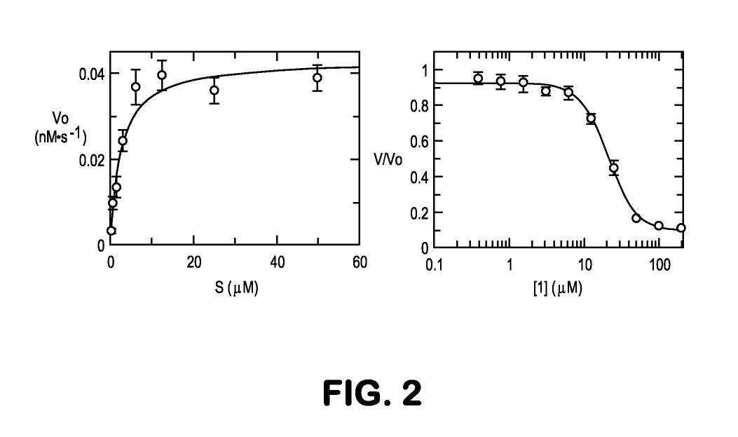

Selection of monoclonal catalytic antibodies (mAbs) was performed by HPLC detection of des-octanoyl ghrelin formation upon incubation of synthetic native rat ghrelin. The initial screen for catalysis for the hydrolysis of rat ghrelin to its des-octanoyl form by the panel of mAbs indicated that several antibodies could accelerate the hydrolysis. From this initial screen 3 mAbs demonstrated turn-over and were evaluated in greater detail. To perform expedient, safe and reliable kinetic analysis of the selected antibodies a new substrate 3 and des-octanoyl product 4 were designed (FIG. 1), based on the following criteria: (i) ghrelin's extinction coefficient is quite poor and impedes HPLC analysis at low concentration and (ii) the only alternative currently available that grants sensitive detection is ¹²⁵I-labeled ghrelin. Thus, 3 and 4 were prepared so as not to compromise the “wild-type” structure of ghrelin to increase sensitivity. Using compounds 3 and 4 in HPLC assay allowed us to identify the most proficient of the initially selected antibodies, of which mAb GHR-11E11 was assessed in detail (FIG. 2). A hybridoma cell line producing this antibody was deposited with the American Type Culture Collection and has been assigned a deposit designation number PTA-120177.

### Example 2

### Specificity and Kinetics of Catalytic Anti-Ghrelin Antibody GHR-11E11

Well-behaved Michaelis-Menten kinetics were observed with GHR-11E11 (K_{M}=2.4 μM, k_{cat}=2.59×10⁻³ min⁻¹, k_{cat}/k_{uncat}=120), which was competitively inhibited by transition state analog 1 (Kᵢ=0.14 μM—this value was calculated from the observed IC₅₀ value, using a fixed substrate concentration and varying inhibitor concentration). The thermodynamic Kᵢ was determined from K_{i,app} via the relationship for competitive kinetics: Kᵢ=K_{i,app}/(1+[S]/K_{M}), where [S]=400 μM and K_{M}=2.4 μM. This is the first example to our knowledge of antibody-catalyzed hydrolysis of an aliphatic long-chain alkyl ester. Interestingly, although turnover numbers were modest it appears more sophisticated chemistry in the antibody-combining site likely comes into play because the K_{M}/Kᵢ≈k_{cat}/k_{uncat} relationship, according to a standard thermodynamic cycle based on TS theory, would predict a rate enhancement for mAb GHR-11E11 of only ≈15, 8-fold less than that actually observed (see FIG. 2).

The specificity of the mAb GHR-11E11 was examined using 6 synthetic ghrelin screening substrates 5-10, which included Ser3(butyryl)-, Ser3(acetyl)-, and Ser3(palmitoyl)-ghrelin-Mca peptides, and 3 Ser3(octanoyl)-ghrelin-derived peptides with Phe4→Ala4, Ser2→Gly2, and Gly1-Ser2→Met1-Gly2 mutations in the N-terminal amino acid sequence (FIG. 1). The ghrelin peptides with variable length of the fatty acid ester chains were designed to determine the diapason of mAb GHR-11E11 catalytic ability toward hydrolysis of ghrelin ester peptides, whereas the ghrelin peptide analogs were used to establish the specificity of the catalytic mAb for native ghrelin in the presence of other endogenous O-acylated proteins and peptides. In summary, mAb GHR-11E11 was found to accelerate hydrolysis of the Ser3(butyryl)-ghrelin construct 5, and Michaelis-Menten kinetics were observed (KM=8.49 μM, kcat=4.57×10-3 min-1, kcat/KM=9 M-1·s-1). This result is in line with the GHR-11E11-catalyzed hydrolysis kinetics of the Ser3(octanoyl)-ghrelin substrate 3, because this antibody was secured with a butylphosphonate ester hapten. At the same time, OHR-11E11 did not substantially alter the hydrolysis rates of the Ser3(acetyl)- and Ser3(palmitoyl)-ghrelin peptide analogs 6 and 7. Catalytic antibody GHR-11E11 catalyzes the hydrolysis of ghrelin peptide ester analogs with a narrow range of the O-acyl group length, which includes both Ser3(butiryl)- and Ser3(octanoyl)-ghrelins, but the catalytic activity of the antibody drops rather abruptly with a shorter O-acyl group, and diminishes more gradually with increasing length of the O-acyl group. Furthermore, GHR-11E11 showed little ability to significantly catalyze hydrolysis of the ghrelin peptide analogs 8-10, which suggests that this antibody is highly specific for the amino acid sequence of native ghrelin, and is unlikely to affect other endogenous O-acylated proteins and peptides.

### Example 3

### Catalytic Activity of Anti-Ghrelin Antibody GHR-11E11

The observed catalytic efficiency of antibody GHR-11E11 (kcat/KM=18 M-1·s-1) is modest; however, because of ghrelin's short half-life in mammals and because circulating plasma ghrelin concentrations have been estimated to be subnanomolar, a high catalytic proficiency may not be necessary to be of potential in vivo functional relevance. To determine the catalytic activity of GHR-11E11 in vivo, adult male C57BL/6J mice were administered GHR-11E11 (n=4) or a control Ab (an anti-nicotine Ab; NIC-1 9D9, n=5) intravenously by tail vein. Blood was collected from the submandibular vein into chilled polypropylene tubes containing EDTA, PMSF, and HCl to reduce degradation or desoctanoylation of ghrelin. As expected, baseline plasma levels of acylated (119.6±24.6 vs. 93.3±15.6 pg/mL) and des-acyl ghrelin (743.1±86.8 vs. 680.7±81.0 pg/mL), measured by specific ELISAs (BioVendor), did not differ between groups. However, 15 min after treatment, acylated ghrelin levels decreased by 90±18% in mice treated with GHR-11E11 (P<0.05 0%), a reduction not seen in control mice (23.3±25.1 vs. 135.1±35.0 pg/mL, respectively, P<0.05) or in levels of des-acyl ghrelin (768.4±79.0 vs. 635.3±130.3 pg/mL). As a result, the ratio of acylated/des-acyl ghrelin was significantly lower in mice, which received the catalytic anti-ghrelin antibody (0.025±0.039 vs. 0.186±0.052, P<0.05). Acylated, but not des-acyl, ghrelin levels also tended to be lower 24-h after administration in GHR-11E11-treated mice than in controls (103.5±9.6 vs. 133.3±12.1 pg/mL, respectively, P=0.07). Interestingly, in vitro studies demonstrated that the catalytic activity of GHR-11E11 was entirely abrogated in the presence of 5 μM serine esterase inhibitor PMSF, in agreement with similar literature precedents, also indicating that the antibody-induced reduction in acylated ghrelin levels occurred in vivo, rather than after blood collection.

Since human ghrelin and rat ghrelin share substantial sequence identity (differing only at positions 11 and 12), this antibody would have comparable catalytic activity against human ghrelin and other ghrelin proteins with similar structures.

### Example 4

### Antibody GHR-11E11 Increases Energy Expenditure and Reduces Food Intake

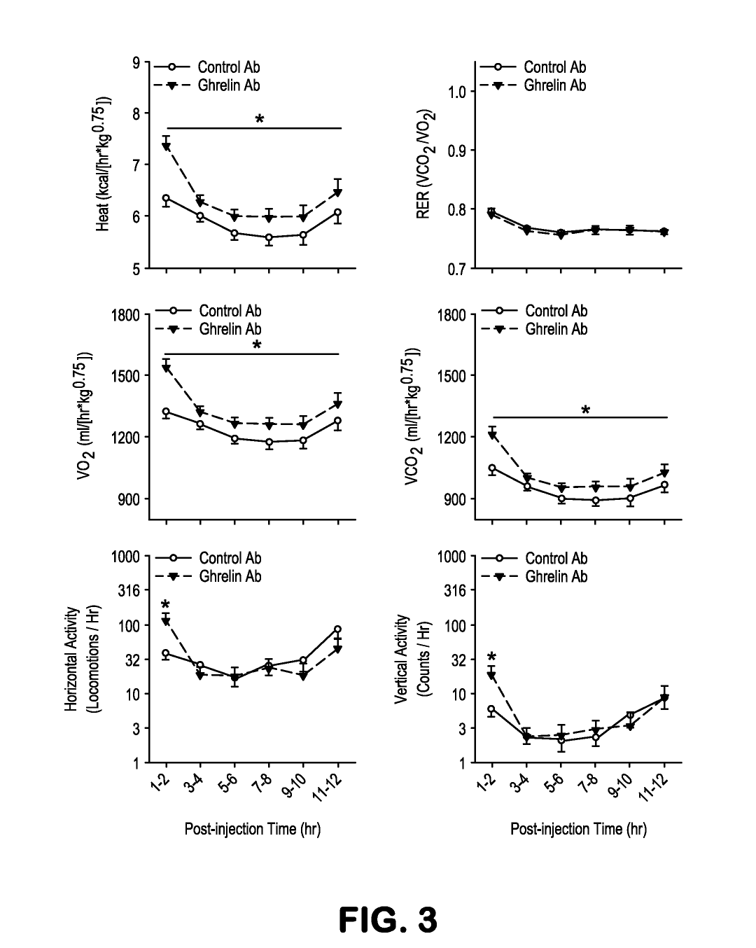

We sought to determine whether i.v. administration of GHR-11E11 altered metabolic rate or refeeding in fasting mice, in which ghrelin activity is increased. Adult male C57BL/6J mice were surgically implanted with i.v. jugular catheters and allowed to recover. Then, mice were acclimated to individual open-circuit indirect calorimetry chambers equipped with computer-monitored food and water access and with photobeams to detect locomotor activity (Comprehensive Lab Animal Monitoring System, Columbus, Ohio) for at least 72 h. Matched for body weight (25.2±0.5 vs. 25.2±0.6 g), mice were intravenously administered (50 mg/kg) either the catalytic ghrelin Ab (GHR-11E11, n=8) or the anti-nicotine control Ab (n=9) within the first hour of the light cycle. Mice were then subjected to a 24-h fast, during which changes in metabolic rate and locomotor activity were monitored for 12 h. FIG. 3 shows that fasted ghrelin Ab-treated mice expended more energy (“heat”) across the entire light cycle than did fasted mice treated with the control Ab (F[1,14]=20.90, P<0.001). Increased energy expenditure was reflected in increased oxygen consumption (VO2; F[1,14]=22.57, P<0.001) and carbon dioxide production (VCO2; F[1,14]=11.98, P<0.005). Groups did not differ in their relative energy substrate utilization, with values of the respiratory exchange ratio (RER 0.75) indicating greater utilization of lipid than carbohydrate in both groups, as expected from a period of fasting during the light cycle. Ghrelin Ab-treated mice showed more motor activity than controls during the first 2 h after treatment, but not thereafter (Hour×Treatment: F(5, 70)=5.94, P<0.03), the latter finding suggesting that differences in energy expenditure were at least partly independent from increased motor activity.

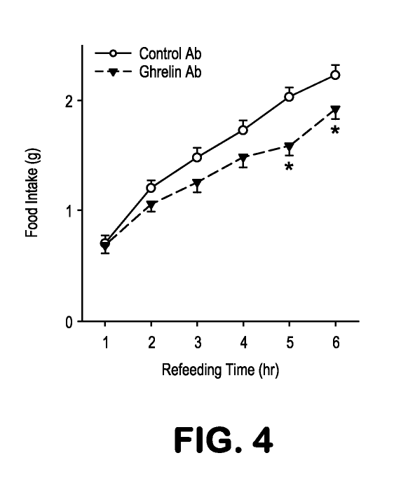

When provided access to chow beginning from the second hour of the next light cycle, mice treated≈24 h earlier with GHR-11E11, the catalytic ghrelin Ab, showed blunted 6-h cumulative food intake (FIG. 4) as compared with mice previously treated with the control nicotine Ab (Treatment×Hour: F[5,60]=6.30, P<0.001).

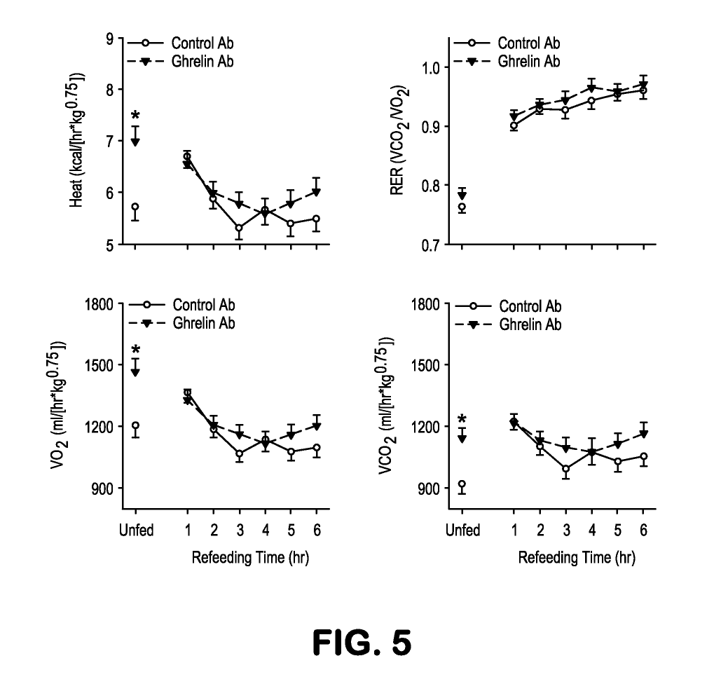

In the hour before they were refed (“Unfed” in FIG. 4, corresponding to the first hour of the light cycle), mice treated with GHR-11E11 showed greater energy expenditure, VO2 and VCO2 than control-Ab treated mice. With refeeding, however, this difference was eliminated; the metabolic rate of refed, control Ab-treated mice rapidly rose to that of ghrelin Ab-treated mice. Refed groups also did not differ in their relative energy substrate utilization, with values of the respiratory exchange ratio rising to levels (RER≈0.9-0.96) indicating greater carbohydrate than lipid utilization in both treatment groups, as expected from a period of refeeding (FIG. 5). Neither vertical nor horizontal motor activity of treated groups differed from one another (data not shown).

All publications, databases, GenBank sequences, patents, and patent applications cited in this specification are herein incorporated by reference as if each was specifically and individually indicated to be incorporated by reference.

What is claimed is:

1. An antibody comprising heavy and light chains, each having a complementarity determining regions (CDRs) of the antibody produced by the hybridoma cell line with ATCC™ deposit number PTA-120177 or each having at least 95% amino acid sequence homology to the CDRs of the antibody produced by the hybridoma cell line with ATCC™ deposit number PTA-120177.

2. The antibody or antigen-binding molecule of claim 1 which is a scFv fragment, an Fv fragment, an Fd fragment, an Fab fragment or an F(ab′)₂ fragment.

3. An isolated or recombinant polynucleotide which encodes a polypeptide comprising the variable region of the heavy chain or the variable region of the light chain of the antibody of claim 1.

4. A hybrid cell line which produces a monoclonal antibody, the monoclonal antibody being specifically reactive with ghrelin and has the specificity of the antibody produced by hybridoma cell line with ATCC™ deposit number PTA-120177.

5. The cell line of claim 4, wherein the antibody is a catalytic antibody capable of degrading ghrelin.

6. The cell line of claim 4, which has ATCC™ deposit number PTA-120177.

7. A pharmaceutical composition comprising a therapeutically effective amount of the antibody of claim 1 and a pharmaceutically acceptable vehicle.

8. The pharmaceutical composition of claim 7, wherein the antibody is produced by hybridoma cell line with ATCC™ deposit number PTA-120177.

9. The pharmaceutical composition of claim 7, wherein the antibody is a scFv fragment, an Fv fragment, an Fd fragment, an Fab fragment or an F(ab′)₂ fragment.

10. A method of reducing weight or slowing weight gain in a subject, comprising administering to the subject a pharmaceutical composition comprising an antibody comprising heavy and light chains, each having a complementarity determining regions (CDRs) of the antibody produced by the hybridoma cell line with ATCC™ deposit number PTA-120177 or each having at least 95% amino acid sequence homology to the CDRs of the antibody produced by the hybridoma cell line with ATCC™ deposit number PTA-120177, thereby reducing weight or slowing weight gain in the subject.

11. A method of treating obesity in a subject, comprising administering to the subject a pharmaceutical composition comprising An antibody comprising heavy and light chains, each having a complementarity determining regions (CDRs) of the antibody produced by the hybridoma cell line with ATCC™ deposit number PTA-120177 or each having at least 95% amino acid sequence homology to the CDRs of the antibody produced by the hybridoma cell line with ATCC™ deposit number PTA-120177, thereby treating obesity in the subject.
